(19) United States
(12) Patent Application Publication
(10) Pub. No.: US 2002/0019344 A1
(43) Pub. Date: Feb. 14, 2002

(54) Methods of preventing allograft rejection

(76) Inventor: Harrihar A. Pershadsingh, Bakersfield, CA (US)

Correspondence Address: TOWNSEND AND TOWNSEND AND CREW, TWO EMBARCADERO CENTER, EIGHTH FLOOR, SAN FRANCISCO, CA, 94111-3834, US

(21) Appl. No.: 09/795,043

(22) Filed: Feb. 26, 2001

Related U.S. Application Data
(60) Provisional application No. 60/185,347, filed on Feb. 26, 2000.

(51) Int. Cl. A61K 38/13; A61K 31/66; A61K 31/573; A61K 31/522; A61K 31/4741

(52) U.S. Cl. 514/9; 514/291; 514/369; 514/179; 514/261; 514/105; 514/532; 514/534

(57) ABSTRACT

Methods of promoting allograft survival, by attenuating or preventing allograft rejection, and treating or ameliorating the post-transplantation syndrome complex associated with allograft rejection and immunosuppressive pharmacotherapy used to prevent allograft rejection, are described. These methods comprise administering to an human or animal in need of treatment an effective amount of an insulin-sensitizing compound or and pharmaceutically acceptable salts and solvates thereof, administered alone or in combination with other immunosuppressive drugs.

### CROSS-REFERENCES TO RELATED APPLICATIONS

This application claims benefit under 35 U.S.C. §119(e) of provisional application U.S. Ser. No. 60/185,347 filed one Feb. 26, 2000, which is herein incorporated by reference in its entirety for all purposes.

### FIELD OF THE INVENTION

This invention relates to disorders involving inflammatory and immunomodulatory responses of the immune system which are triggered by transplantation of allogeneic organs or tissues and the use of immunosuppresive therapy to prevent rejection of the allograft. The invention relates to new methods for preventing acute and chronic allograft rejection and promoting graft survival and maintenance, and to ameliorating or preventing post-transplantation adverse clinical effects associated with allograft rejection. In another aspect, the invention relates to the treatment and amelioration of adverse clinical conditions or diseases related to immunosuppressive pharmacotherapy used to treat or prevent allograft rejection. This invention also relates to methods for screening libraries of compounds to determine which are likely candidates for use in the practice of this invention.

### BACKGROUND OF THE INVENTION

The immunomodulatory/immunological mechanisms underlying organ transplant rejection are not well understood. Immunosuppressive agents capable of blocking various steps of the immune response have been utilized to prevent the rejection of allografts and promote their survival (Gorantla, V. S., Microsurgery, 20:420-9 (2000)). Organ transplantation is now common. For patients with end-stage renal, cardiac, hepatic, or pulmonary failure, it can be lifesaving.

Over the past two decades, the development of new immunosuppressive drugs with improved efficacy and decreased toxicity has led to substantial improvement in the survival of patients and in short-term graft survival for all organs (Sayegh, M. H. et al., New Eng J Med, 338:1813-21 (1998)). Much of this improvement can be attributed to better prophylaxis against and treatment for acute rejection, an immune response against the graft that usually occurs within the first six months after transplantation (Platt, J. L. Transplant Proc 32:839-40 (2000)). Despite the improvement in short-term results, the long-term survival of grafts that are functional at one year has changed little. The cause of late graft loss is usually chronic rejection, a poorly understood disorder that may be mediated by both alloantigen-dependent and alloantigen-independent mechanisms (Vazquez, M. A. Am J Med Sci, 320:43-58 (2000)).

Several factors have been implicated in autoimmune disease and the development of chronic rejection. They include a chronic low-level host immune response to alloantigens expressed by the graft, early episodes of acute rejection, and side effects of current medications (such as cyclosporine nephrotoxicity and hyperlipidemia). Although many cells can participate in the process of acute transplant rejection, only T lymphocytes appear to be absolutely required (Gorantla, V. S., Microsurgery, 20:420-9 (2000); Sayegh, M. H. et al., New Eng J Med, 338:1813-21 (1998)). This class of lymphocytes consists of functionally and phenotypically distinct populations, the best characterized of which are helper T cells and cytotoxic T cells. Activation of various T lymphocyte subpopulations occur via costimulatory pathways which involve MHC molecules, T-cell antigen receptors, interaction of a wide variety of cytokines or cytokine antagonists (interferon-gamma, interleukin (IL)-2, IL-4, IL-5, IL-6 and IL-10) and adhesion molecules. Most immune responses depend on the activation of T cells (Gorantla, V. S., Microsurgery, 20:420-9 (2000); Sayegh, M. H. et al., New Eng J Med, 338:1813-21 (1998)). However, the mechanisms whereby these pathways activate T lymphocytes and modulate the immune response are poorly understood.

Medical therapy primarily centers on the suppression of inflammation and blockade of costimulatory pathways of T-cell activation (Gorantla, V. S., Microsurgery, 20:420-9 (2000); Sayegh, M. H. et al., New Eng J Med, 338:1813-21 (1998); Gonin, J. M. Adv Ren Replace Ther., 7:95-116 (2000)). These include immunosuppressive glucocorticoids (e.g. prednisone), inhibitors of purine synthesis (e.g. azathioprine, cyclophosphamide, mycophenolate mofetil), macrolide immunophilin modulators (e.g. sirolimus), and inhibitors of the calcineurin-dependent cytokine synthesis in activated lymphocytes (e.g. cyclosporine, ASM 981, tacrolimus). The macrolide immunophilin modulators and calcineurin inhibitors are especially useful in preventing severe, refractory transplant rejection, but these drugs are nephrotoxic, and can induce hypertension and hyperlipidaemia. Hyperlipidemia is extremely common following transplantation and very likely contributes to the high mortality from cardiovascular disease. Post-transplantation hyperlipidemia is characterized by increased or unchanged serum levels of triglyceride and high-density lipoprotein (HDL) and increased levels of low-density lipoprotein (LDL) and total cholesterol. In some categories of patients, a higher incidence of de-novo diabetes mellitus is seen with tacrolimus. Chronic prednisone therapy can lead to glucocorticoid excess (Cushing's syndrome), insulin resistance resulting in type 2 diabetes, and obesity, hypertension and hyperlipidemia resulting in cardiovascular disease, and osteopenia and osteoporosis resulting in bone fractures (Rao, V. K. Surg Clin North Am, 78:113-32 (1998)). Moreover, these therapies are not uniformly successful in all patients and many patients experience acute and/or chronic allograft rejection, or they become recalcitrant to therapy (Platt, J. L. Transplant Proc 32:839-40 (2000); Vazquez, M. A. Am J Med Sci, 320:43-58 (2000)).

The peroxisome proliferator activated receptors (PPARs) are nuclear transcription factors that exist as three isoforms, alpha, beta and gamma, and belong to the nuclear receptor superfamily that includes receptors for the steroid hormones, thyroid hormone, vitamin D and the retinoids (Mangelsdorf, D. J. et al., Cell, 83:841-50 (1995)). The peroxisome proliferator activated receptor (PPAR)-alpha (PPARalpha) is more or less ubiquititously distributed throughout the mammalian organs and tissues. PPARgamma is expressed to a high degree in tissues (e.g. the spleen) and cellular elements (e.g. T lymphocytes, B lymphocytes, neutrophils, monocyte/macrophages) of the immune system (Braissant, O. et al. Endocrinology, 137:354-66 (1996); Fajas, L. et al. J Biol Chem, 272:18779-89 (1997)). It has recently been demonstrated that activation of human monocyte/macrophages and T lymphocytes is inhibited by PPARgamma agonists (Clark, R. B. et al. J Immunol, 164:1364-71 (2000)). This inhibitory effect occurs by PPARgamma co-association with transcription factor, nuclear factor of activated T cells (NFAT), ultimately resulting in blockade of IL-2 production and secretion (Yang, X. Y. et al. J Biol Chem., 275:4541-4 (2000)). Activation of PPARgamma by structurally unrelated ligands results in the inhibition of proliferative and inflammatory processes in cells at various points in different signal transduction pathways that govern cell growth and immune adaptation (Willson, T. M. et al. J Med Chem., 43:527-50 (2000)).

According to the prior art, allograft rejection may be prevented or treated by decreasing nitric oxide production in association with blocking of T lymphocyte activation, blocking NFAT binding to DNA, and inhibition of IL-2 MRNA transcription and IL-2 production by activated T lymphocytes (Berard, J. L. et al. Pharmacotherapy, 19:1127-37 (1999)). This is thought to occur by increasing nitric oxide production, and has been shown to be the case in heart and kidney transplantation (Albrecht, E. W. et al. Transplantation, 70:1610-6 (2000); Liu, Z. et al. Atherosclerosis, 140:1-14 (1998)). In contrast, PPARgamma activation has been shown to inhibit nitric oxide production (Neve, B. P. et al. Biochem Pharmacol, 60:1245-50 (2000)). Moreover, activation of PPARgamma by troglitazone and rosiglitazone has been shown to inhibit the production of the inflammatory cytokines IL-2, IL-6 and TNF-alpha, whereas the potent PPARgamma agonist AD-5075 had no effect on tumor necrosis factor-alpha (TNF-alpha) and lipopolysaccharide production in monocyte/macrophages (Thieringer, R. et al. J Immunol., 64:1046-54 (2000)). Consequently, PPARgamma ligands would not have been expected to prevent allograft rejection.

PPARgamma exists as at least three subtypes: gamma1, gamma2 and gamma3. According to this invention, administration of compounds that bind to and activate PPARgamma (defined as any subtype or combination of subtypes including those not yet discovered) or PPARalpha or a compound that is a dual activator (co-activator) of PPARgamma and PPARalpha, or a compound that activates PPARgamma/RXR heterodimers as molecular targets in the treatment or prevention or amelioration of allograft rejection or related clinical complications thereof This invention further applies to the downregulation of pro-inflammatory nuclear factors (e.g. NF-kappaB, AP-1, NFAT) provided by the compounds of this invention, as novel methods for treating and preventing allograft rejection and inflammatory and proliferative conditions or diseases associated with allograft transplantation.

In addition to PPARgamma activation, antiproliferative and anti-inflammatory effects are mediated through a number of other mechanisms, including inhibition of mitogen-activated protein kinase (MAP kinase), inhibition of protein kinase C (PKC), inhibition of agonist (growth factor or hornone)-induced calcium entry. The thiazolidinediones are PPARgamma ligands that have anti-inflammatory effects which are mediated primarily through inhibition of the activation of nuclear transcription factors that promote expression of inflammatory cytokines, especially NF-kappaB, AP-1 and NFAT. Prostaglandin J2 derivatives have been shown to bind and activate PPARgamma and to exert anti-inflammatory effects by inhibiting nuclear factor-kappaB (NF-kappaB), activated protein-1 (AP-1) and NFAT. However, the prior art does not demonstrate that the inhibitory effects of prostaglandin J2 derivatives are mediated only by binding or activating PPARgamma. Prostaglandins and thiazolidinediones have many different effects on cell function that are not mediated by PPARgamma and therefore, the prior art does not enable one to predict whether non-prostaglandin compounds that bind or activate PPARgamma will also inhibit activation of NF-kappaB, NFAT and AP-1.

### BRIEF SUMMARY OF THE INVENTION

This invention provides for improvement over existing practice in that drugs encompassed by the current invention do not, or rarely, have the debilitating or unpleasant side effects as seen with current medical therapies for treating or preventing allograft rejection and the metabolic, inflammatory and proliferative conditions or diseases associated with allograft rejection, and the administration of immunosuppressive therapy used to promote allograft survival. Specifically, the invention relates to the use of PPARgamma activators, or dual PPARgamma and PPARalpha co-activators, or activators of PPARgamma/RXR heterodimers in the prevention and treatment of allograft rejection and for treating the pathological conditions associated with allograft rejection and the immnunosuppresive therapy used to prevent the allograft rejection and promote graft survival.

Examples of drugs that bind to or modify the activity of PPARgamma include thiazolidinediones such as troglitazone (Sankyo/Parke-Davis), rosiglitazone (SmithKline Beecham), pioglitazone (Upjohn & Pharmacia), 5-aryl-2,4-thiazolidinedione derivatives (Merck, US Pat. No. 6,008,237), AD-5075 (Merck Research Laboratories, Rahway, N.J.), alpha-methoxy-beta-phenyl propanoic acid derivatives (SmithKline Beecham), N-(2-Benzoylphenyl)-L-tyrosine derivatives (GlaxoWellcome), phenylacetic acid derivatives (L-165,041, L-165,461, L-796,449 AD-5075 (Merck, U.S. Pat. No. 5,859,051), PPARgamma-selective prostaglandin or prostaglandin-like compounds (Salk Institute, U.S. Pat. No. 6,022,897).

Another aspect of this invention relates to the use of compounds that bind or modify the activity of PPARgamma can also be given in combination with other immunosuppressive compounds to provide for a synergistic effect in the treatment or prevention of inflammatory and proliferative conditions or diseases associated with allograft transplantation. Examples of such compounds that provide for synergistic effect when given in combination with the drugs encompassed by the current invention include ligands for the glucocorticoid nuclear receptor ligand (e.g. prednisone), inhibitors of purine synthesis (e.g. azathioprine, cyclophosphamide, mycophenolate), and inhibitors of the calcineurin-dependent cytokine synthesis in activated lymphocytes (e.g. cyclosporine, tacrolimus), and the macrolide, sirolimus. Another aspect of this invention relates to the use of RXR/PPARgamma agonists (e.g. LG100754).

### DETAILED DESCRIPTION OF THE INVENTION

The current invention involves the discovery that compounds that bind to and activate PPARgamma (any subtype or combination of subtypes), or a compound that activates both PPARgamma and PPARalpha, or a compound that activates the PPARgamma/RXR heterodimer, are useful for: 1) preventing and treating acute and chronic allograft rejection by promoting graft survival, 2) attenuating or preventing the metabolic, inflammatory and proliferative conditions or diseases associated with allograft transplantation, and 3) attenuating or preventing the metabolic, inflammatory and proliferative conditions or diseases associated with immunosuppressive therapy used to prevent allograft rejection and promote allograft survival. Examples of drugs that bind to or modify the activity of PPARgamma and are useful for this purpose include thiazolidinediones such as troglitazone, pioglitazone, rosiglitazone, 5-aryl-2,4-thiazolidinedione derivatives (Merck, U.S. Pat. No. 6,008,237), and non-thiazolidinediones such as alpha-methoxy-beta-phenyl propanoic acid derivatives (Haigh et al. Bioorg Med Chem 1999; 7:821-30), N-(2-Benzoylphenyl)-L-tyrosine derivatives (Henke B R, et al. J Med Chem. 1998;41:5020-36), indole-based PPARgamma agonist (Henke B R, et al. Bioorg Med Chem Lett. 1999;9:3329-34, phenylacetic acid derivatives Merck, U.S. Pat. No. 5,859,051), PPARgamma-selective prostaglandins such as the cyclopentenone prostaglandins belonging to the A1 and J2 series and their metabolites (15-deoxy-prostaglandin A1, 15-deoxy-prostaglandin J2, 15-deoxy-12,14-prostaglandin J2) or prostaglandin-like compounds (U.S. Pat. No. 6,022,897). Ligands that have utility and efficacy in the practice of this invention include PPARgamma agonists (e.g. the thiazolidinediones), partial PPARgamma agonists (MCC-555, JTT-501), and PPARgamma/RXR heterodimer agonists (e.g. LGD100754) and fatty acids, eicosanoids, and xenobiotics (Devchand, P. R. et al. Adv Exp Med Biol 469:231-6 (1999)).”

### SOURCE INFORMATION ON COMPOUNDS LISTED IN THIS APPLICATION

| Compound | Reference |
|---|---|
| MCC 555 | 1, 2 |
| 3-substituted benzyl)thiazolidine-2,4-diones, e.g. KRP 297 | 1, 3 |
| Indole-based PPARgamma agonists, e.g. BRL 48482 | 4 |
| 5-aryl-2,4-thiazolidinedione | 5 |
| isoxazolinedione derivatives, e.g. JTT-501 | 1, 6, 7 |
| alpha-methoxy-beta-phenylpropanoic acids, | 1, 8, 9 |
| e.g. SB 236636 | |
| N-(2-Benzoylphenyl)-L-tyrosine derivative, e.g. | 1, 10, 11, 12 |
| G1262570, GW7845 | |
| Phenylacetic acid derivatives, e.g. L-764486 | 13, 14 |
| LG100268, LGD1069, LG100268, LGD100324, | 1, 15 |
| LG100754 | |
| SDZ-RAD is the 40-O-(2-hydroxyethyl) derivative of | 16 |
| sirolimus (rapamycin) | |
| GW 409544, GW 501516, GI 181771 | 1, 10, 11, 12 |
| CI-1037/CS011 | 17, 18 |
| CLX-0901, CLX-0921, CLX-0940 | 19, 20, 21 |
| NIP-221, NIP-223 | 22, 23 |

### REFERENCES

1. Willson T M, et al. The PPARs: from orphan receptors to drug discovery. J Med Chem. 2000; 43:527-50.

2. Prabhakar C, et al. Synthesis and biological activity of novel thiazolidinediones. Bioorg Med Chem Lett 1998; 8:2725-30.

3. Nomura M, et al. (3-substituted benzyl)thiazolidine-2,4-diones as structurally new antihyperglycemic agents. Bioorg Med Chem Lett 1999; 9:533-8.

4. Henke B R, et al. Synthesis and biological activity of a novel series of indole-derived PPARgamma agonists. Bioorg Med Chem Lett 1999; 9:3329-34.

5. U.S. Pat. No. 6,008,237 Arylthiazolidinedione derivatives.

6. U.S. Pat. No. 6,057,343 Isoxazolidinedione compounds and use thereof.

7. U.S. Pat. No. 6,037,359 Isoxazolidinedione derivatives and use thereof.

8. Buckle D R, et al. A. Non-thiazolidinedione antihyperglycemic agents. 1: R-Heteroatom substituted alpha-phenylpropanoic acids. Bioorg Med Chem Lett 1996;

6:2121-6.

9. Buckle D R, et al. A. Non-thiazolidinedione antihyperglycemic agents. 2: R-Carbon substituted alpha-phenylpropanoic acids. Bioorg Med Chem Lett 1996; 6:2127-30.

10. Henke B R, et al. N-(2-Benzoylphenyl)-L-tyrosine PPARgamma agonists. 1. Discovery of a novel series of potent antihyperglycemic and antihyperlipidemic agents. J Med Chem 1998; 41:5020-36.

11. Collins J L, et al. N-(2-Benzoylphenyl)-L-tyrosine PPARgamma agonists. 2. Structure-activity relationship and optimization of the phenyl alkyl ether moiety. J Med Chem 1998; 41:5037-54.

12. Cobb J E, et al. N-(2-Benzoylphenyl)-L-tyrosine PPARgamma agonists. 3. Structure-activity relationship and optimization of the N-aryl substituent. J Med Chem 1998;

41:5055-69.

13. U.S. Pat. No. 6,090,836 Benzisoxazole-derived antidiabetic compounds.

14. Berger J, et al. Novel peroxisome proliferator-activated receptor (PPAR) gamma and PPARdelta ligands produce distinct biological effects. J Biol Chem 1999;

274:6718-25.

15. Hamann L G. An efficient, stereospecific synthesis of the dimer-selective retinoid X receptor modulator (2E,4E,6Z)-7-[5,6,7,8-tetrahydro-5,5,8,8-tetramethyl-2-(n-propyloxy) naphthalen-3-yl]-3-methyl octa-2,4,6-trienoic acid. J Org Chem 2000; 65:3233-5.

16. Viklicky O, et al. SDZ-RAD prevents manifestation of chronic rejection in rat renal allografts. Transplantation 2000; 69:497-502.

17. Kazushiaraki, M, et al. Antidiabetic characterization of CS-011: a new thiazolidinedione with potent insulin-sensitizing activity. Diabetes, 2000, Abstracts 425 and 425A.

18. Chemical name: (±)-5-[4-(6-methoxy-1-H-benzimidazol-2-ylmethoxy)benzyl] thiazoline-2,4-dione hydrochloride

19. Deben D, et al. Mechanism of CLX-0901 action: a novel plant-derived orally active anti-diabetic compound. Diabetes 2000; 49(Suppl.1), Abstract Nos: 414 and 414A.

20. Satya M, et al. CLX-0921: A new PPAR-g agonist antidiabetic thiazolidinedione compound. Diabetes 2000; 49(Suppl.1), Abstract Nos: 475 and 475A.

21. Partha N, et al. CLX-0940: A non-thiazolidinedione PPAR agonist and insulin sensitizer. Diabetes 2000; 49(Suppl.1), Abstract No: 1519.

22. Takeshi N, et al. NIP-221 Is a novel and potent thiazolidinedione-based PPARgamma ligand with long biological half-time. Diabetes 2000; 49(Suppl.1), Abstract No: 480.

23. Takeshi N, et al. NIP-223 Is a novel and potent thiazolidinedione-based PPARgamma ligand. Diabetes 2000; 49(Suppl.1), Abstract No: 481.

24. Reginato M J, et al. A potent antidiabetic thiazolidinedione with unique peroxisome proliferator-activated receptor gamma-activating properties. J Biol Chem. 1998; 273:32679-84.

This invention provides for improvement over existing practice in that drugs encompassed by the current invention do not, or rarely have the debilitating or unpleasant side effects of current medical therapies for preventing or treating metabolic, inflammatory and proliferative conditions or diseases associated with allograft transplantation and the rejection process, and the use of pharmacotherapy to promote and maintain allograft survival post-transplantation. Examples of inflammatory, proliferative, metabolic and degenerative conditions or diseases associated with allograft transplantation and pharmacological immune suppression include, but are not limited to: acute allograft rejection, chronic allograft rejection, graft versus host disease, post-transplantation de novo malignancy (e.g. lymphoma and epidermal cancers), osteoporosis, osteopenia, hyperlipidemia, insulin resistance, type 2 diabetes, hypertension, atherosclerosis, endarteritis, glomerulonephritis, vasculopathy, cardiomyopathy and congestive heart failure.

The term PPARgamma is used throughout this entire writing to mean any PPARgamma subtype or any combination thereof PPARgamma subtypes include PPARgamma1, PPARgamma2 and PPARgarnma3. The term PPARgamma/PPARalpha is used throughout this entire writing to mean, in general, any compound that is a dual activator of both PPARgamma and PPARalpha. The term PPARgamma/PPARalpha is used throughout this entire writing to mean, in particular, any compound that is PPARgamma/PPARalpha co-activator, wherein the ED50 for PPARgamma activation is within 1 to 2 orders of magnitude of the ED50 for PPARalpha activation.

Another aspect of this invention relates to the use of compounds that activate PPARgamma, both PPARgamma and PPARalpha, or PPARgamma/RXR heterodimers, and can be given orally or intravenously in combination with other immunosuppressive compounds to provide for a synergistic effect in the treatment or prevention of allograft rejection and/or inflammatory and proliferative diseases involved in syndrome complex of allograft rejection, adverse effects associated with immunosuppressive pharmacotherapy, and other transplantation-related diseases. This enables the physician to administer lower doses of immunosuppresive compounds and thereby decrease the untoward side effects associated with administration of such drugs.

Examples of such compounds that provide for synergistic effect when given in combination with the drugs encompassed by the current invention include ligands for the glucocorticoid nuclear receptor ligand (e.g. prednisone), inhibitors of purine synthesis (e.g. azathioprine and mycophenolate), immunoplilin modulators such as inhibitors of the calcineurin-dependent cytokine synthesis in activated lymphocytes (e.g. cyclosporine, tacrolimus), and the macrolide antibiotic sirolimus.

Another aspect of this invention relates to the use of RXR/PPARgamma ligands known as “rexinoids”, including but not limited to compounds such as LG100268, LGD100324, LG100754. A preferred dosage range for administration of an RXR or PPARgamma/RXR rexinoid ligand would typically be from 0.1 to 100 mg per square-meter of body surface area, depending on the drug's ability to bind to or modify the activity of its cognate nuclear receptor, given in single or divided doses, orally or by continuous infusion, two or three times per day.

The terms treatment and prevention include their usually accepted meanings and include treating a human subject to decrease clinical symptoms of allograft rejection and ameliorate or prevent the metabolic, inflammatory and proliferative pathological conditions or diseases associated with allograft transplantation, and ameliorate or decrease or prevent the adverse clinical conditions or diseases associated with the administration of immunosuppressive therapy used to prevent allograft rejection and promote allograft survival, or to prevent relapses in patients exhibiting these diseases or conditions. The present method includes both medical therapeutic and/or prophylactic treatment, as necessary.

The term insulin-sensitizing agent means a pharmacological decreases insulin resistance and the associated hyperinsulinemia. The terms synergism or synergistic activity include their usually accepted meanings. For example, a compound of this invention, when used in combination with an immunosuppressive agent presently approved or recommended for use in preventing allograft rejection, such as the glucocorticoid prednisone, would require a lower dose (of prednisone) to achieve the effect of a standard therapeutic dose for preventing allograft rejection. Consequently, the improvement over existing practice is the use of an insulin-sensitizer (one of the subject compounds) in combination with a lower dose of another immunosupressive drug (e.g. prednisone, cyclosporine, tacrolimus, sirolimus), in order to oppose the metabolic defects (e.g. insulin resistance), hyperlipidemia, and other adverse cardiovascular effects (e.g. hypertension, atherosclerosis, renal impairment) associated with these immunosuppressive drugs. In a functional test to define an insulin-sensitizing agent, it is a compound that: 1) induces differentiation of adipocytes in vitro, and 2) lowers blood insulin concentration in an animal or human with insulin resistance and hyperinsulinemia, in vivo.

Using a method of the invention, therapeutic compounds are typically administered to human patients orally, intra-muscularly, intravascularly, and/or subcutaneously. Preferred methods of delivery are via oral or vascular routes. Preferably, the compositions are administered in unit dosage forms suitable for single administration of precise dosage amounts.

For oral administration, either solid or fluid unit dosage forms can be prepared. For preparing solid compositions such as tablets, the compound of interest is mixed into formulations with conventional ingredients such as talc, magnesium stearate, dicalcium phosphate, magnesium aluminum silicate, calcium sulfate, starch, lactose, acacia, methylcellulose, and functionally similar materials as pharmaceutical diluents or carriers. Capsules are prepared by mixing the compound of interest with an inert pharmaceutical diluent and filling the mixture into a hard gelatin capsule of appropriate size. Soft gelatin capsules are prepared by machine encapsulation of a slurry of the compound of interest with an acceptable vegetable oil, light liquid petrolatum or other inert oil. Fluid unit dosage forms for oral administration such as syrups, elixirs and suspensions can be prepared. The water soluble forms can be dissolved in an aqueous vehicle together with sugar, aromatic flavoring agents and preservatives to form a syrup. An elixir is prepared by using a hydroalcoholic (e.g., ethanol) vehicle with suitable sweeteners such as sugar and saccharin, together with an aromatic flavoring agent. Suspensions can be prepared with an aqueous vehicle with the aid of a suspending agent such as acacia, tragacanth, methylcellulose and the like. Appropriate formulations for parenteral use are apparent to the practitioner of ordinary skill. Usually, the therapeutic compound is prepared in an aqueous solution (discussed below) in a concentration of from about 1 to about 100 mg/ml. More typically, the concentration is from about 10 to 60 mg/ml or about 20 mg/ml. The formulation, which is sterile, is suitable for various parenteral routes including intramuscular, intravascular, and subcutaneous.

An alternative parenteral route of administration is intravascularly via hypodermic injection or by intravascular catheter. The drug may be introduced intravascularly directly as a solution, suspension or encapsulated in microspheres, single or multilamellar vesicles, or covalently attached to a polymeric delivery vehicle such as polyethylene glycol.

The amount of the therapeutic compound to be administered and the compound's concentration in the local formulations depend upon the vehicle selected, the clinical condition of the patient, the side effects and the stability of the compound in the formulation. Thus, the physician employs the appropriate preparation containing the appropriate concentration of the therapeutic compound and selects the amount of formulation administered, depending upon clinical experience with the patient in question or with similar patients.

In addition to the therapeutic compound, the compositions for various modes of administration may include, depending on the formulation desired, pharmaceutically-acceptable, non-toxic carriers or diluents, which include vehicles commonly used to form pharmaceutical compositions for animal or human administration. The diluent is selected so as not to unduly affect the biological activity of the combination. Examples of such diluents which are especially useful for injectable formulations are water, the various saline solutions, Ringer's solution, dextrose solution, and Hank's solution. In addition, the pharmaceutical composition or formulation may include additives such as other carriers; adjuvants; or nontoxic, nontherapeutic, nonimmunogenic stabilizers and the like.

Furthermore, excipients can be included in the formulation. Examples include cosolvents, surfactants, oils, humectants, emollients, preservatives, stabilizers and antioxidants. Any pharmacologically acceptable buffer may be used, e.g., tris or phosphate buffers. Effective amounts of diluents, additives and excipients are those amounts which are effective to obtain a pharmaceutically acceptable formulation in terms of solubility, biological activity, etc. The term “unit dosage form” refers to physically discrete units suitable as unitary dosages for human subjects and animals, each unit containing a predetermined quantity of active material calculated to produce the desired pharmaceutical effect in association with the required pharmaceutical diluent, carrier or vehicle. The specifications for the unit dosage forms of this invention are dictated by and dependent on (a) the unique characteristics of the active material and the particular effect to be achieved and (b) the limitations inherent in the art of compounding such an active material for use in humans and animals. Examples of unit dosage forms are tablets, capsules, pills, powder packets, wafers, suppositories, granules, cachets, teaspoonfuls, tablespoonfuls, dropperfuls, ampoules, vials, aerosols with metered discharges, segregated multiples of any of the foregoing, and other forms as herein described.

Thus, a composition of the invention includes a therapeutic compound which may be formulated with conventional, pharmaceutically acceptable, vehicles for local, oral, or parenteral administration. Formulations may also include small amounts of adjuvants such as buffers and preservatives to maintain isotonicity, physiological and pH stability. Means of preparation, formulation and administration are known to those of skill. See generally Remington's Pharmaceutical Science 15th ed., Mack Publishing Co., Easton, Pa. (1980).

Slow Release Delivery

Slow or extended-release delivery systems, including any of a number of colloids, resins, biopolymers (biological-based systems), systems employing liposomes, and polymeric delivery systems, can be utilized with the compositions described herein to provide a continuous or long term source of therapeutic compound. Such slow release systems are applicable to formulations for use for local administration, in patches, or for oral and parenteral use.

Routes of Administration

In general, the preferred routes of administration are oral, parenteral, or topical. Oral and parenteral administration are preferred in the treatment of the syndrome complex of allograft rejection and other diseases or conditions related to organ transplantation. Local administration may be preferred in some cases.

Dosage and Schedules

An effective quantity of the compound of interest is employed in treatment. The dosage of compounds used in accordance with the invention varies depending on the compound and the condition being treated. For example, the age, weight, and clinical condition of the recipient patient; and the experience and judgment of the clinician or practitioner administering the therapy are among the factors affecting the selected dosage. Other factors include: the route of administration, the patient, the patient's medical history, the severity of the disease process, and the potency of the particular compound. The dose should be sufficient to ameliorate symptoms or signs of the disease treated without producing unacceptable toxicity to the patient. In general, an effective amount of the compound is that which provides either subjective relief of symptoms or an objectively identifiable improvement as noted by the clinician or other qualified observer.

Broadly, for a PPARgamma agonist (e.g. rosiglitazone, pioglitazone, troglitazone) or a PPARgamma/PPARalpha co-activator (e.g., KRP 297, JTT-501, MCC 555), the oral dose is determined from the following formula:

oral dose (in mg)=(k₁)(EC₅₀)(k₂) (LBW)(MW);

wherein k₁, is a dimensionless constant of 5 to 100;

EC₅₀ is the concentration (amount) of compound required to activate or bind to 50% of PPARgamma and 50% of PPARalpha in the sample or patient and is in mole/L units;

k₂ is the fractional water content of the lean body weight (LBW) of the patient =0.72 L/kg, (see, GEIGY SCIENTIFIC TABLES, VOL. 1, Lentner (ed.), p217, Giba-Geigy Ltd., Basle, Switzerland (1981); and

MW is the molecular weight of the compound in g/mole.

For example, troglitazone is a compound encompassed by the methods of this invention. A man with diagnosis of early stage prostate cancer in situ has a lean body weight (LBW) of 70 kg. If k1=10; the EC₅₀ for troglitazone=2.4×10⁻⁶ mol/L and the molecular weight of troglitazone=442 g/mol, then the oral dose in milligrams=(10) (2.4×10⁻⁶ mol/L)(0.72 L/kg×70 kg) (442 g/mol) or 535 mg.

Similarly, an effective dose of rosiglitazone in milligrams for an average man is (10)(0.06×10⁻⁶ mol/L)(0.72L/kg×70kg)(304 g/mole) or 9.2 mg.

Typically, the dosage per day of a thiazolidinedione of this invention will depend on the affinity of the thiazolidinedione for PPARgamma or PPARalpha. The dosages of compounds with high affinity, e.g., rosiglitazone, will range from about 1 mg to about 20 mg, of compounds of intermediate affinity will range from about 20 mg to about 100 mg and compounds with low affinity, e.g., troglitazone, will fall from about 100 mg to about 800 mg. An oral dosing schedule is typically, a single dose once a day. However, more than one dose can be given per day. Because of the lower incidence of undesirable side effects as compared to other immunosuppressive agents, the compounds of this invention can be given until clinical improvement is observed. Once a therapeutic result is achieved, the compound can be tapered or discontinued. Occasionally, side effects warrant discontinuation of therapy.

Because some of the compounds of this invention are to some degree fat-soluble, in a preferred embodiment, the compounds are administered with food. The fats in food provide a lipid micellular phase in which the PPAR gamma modifiers of this invention can solubilize and be more effectively absorbed. Typically, the greater the affinity, the more effective the compound, and the lower the dosage that is an effective amount. The dosage can be administered twice a day, but more or less frequent dosing can be recommended by the clinician.

An effective quantity of the compound of interest is employed in treatment. The dosage of compounds used in accordance with the invention varies depending on the compound and the condition being treated. The age, weight, and clinical condition of the recipient patient; and the experience and judgment of the clinician or practitioner administering the therapy are among the factors affecting the selected dosage. Other factors include the route of administration the patient, the patient's medical history, the severity of the disease process, and the potency of the particular compound. The dose should be sufficient to ameliorate symptoms or signs of the disease treated without producing unacceptable toxicity to the patient.

A preferred dosage range for oral or parenteral administration of a glucocorticoid nuclear receptor ligand, is 1 to 100 mg per square-meter of body surface area depending on the drug's ability to bind to or modify the activity of its cognate nuclear receptor, given in single or divided doses, orally or by continuous infusion, two or three times per day. An example of an effective glucocorticoid is prednisone.

Examples of effective inhibitors of purine synthesis are azathioprine (usually given intravenously or orally) and mycophenolate mofetil (MMF, CellCept(R), usually given orally only. A preferred dosage range for oral or intravenous administration of azathioprine is 1 to 10 mg/kg/day given as a single daily dose on the day of transplantation and post-operatively. A preferred dosage range for oral administration of mycophenolate is 0.25 to 2 grams/day given as divided daily doses every 12 hr, being initiated within 72 hr of transplantation and post-operatively.

Examples of effective immunophilin modulators and inhibitors of the calcineurin-dependent cytokine synthesis in activated lymphocytes are cyclosporine (Sandimmune, Gengraf, Neoral), tacrolimus (Prograf, FK506), sirolimus (RAPA, rapamycin, Rapamune). Preferred methods of administration are intravenous or oral. A preferred dosage range for intravenous and oral of tacrolimus is 0.01 to 0.3 mg/kg/day. Usually tacrolimus is initiated as intravenous therapy and the patient converted to oral therapy as divided daily doses every 12 hr within 2 to 3 days post-operatively.

A broad range of structurally different compounds have been shown to inhibit IL-2 production, and for this reason, useful for preventing allograft rejection. These include the glucocorticoids (e.g. prednisone), macrolide immunophilin modulators (e.g. sirolimus) and inhibitors of the calcineurin-dependent cytokine synthesis in activated lymphocytes (e.g. cyclosporine, ASM 981, tacrolimus).

Yang X Y, et al. (J Biol Chem 2000;275:4541-4) showed that two structurally dissimilar PPARgamma ligands, 15-deoxy Δ-12,14- prostaglandin J2 (15-deoxy-PG J2)and the anti-diabetic drug, troglitazone. The inhibitory effect of troglitazone on IL-2 production was seen at extremely high concentrations, beyond those achievable in the blood therapeutically. Moreover, troglitazone had no effect on IL-2 production at a concentration of 2.5 micromol/L, about 5 times greater than the EC50 (0.55 micromol/L) for transactivation of the PPARgamma receptor (Yang X Y, et al. J Biol Chem 2000;275:4541-4).

Troglitazone is known to act by mechanisms unrelated to PPARgamma activation or its insulin-sensitizing activity. For example, troglitazone inhibits both voltage-dependent calcium currents and capacitative calcium entry into cardiac myocytes (Nakajima T, et al. Circulation 1999; 99:2942-50) and endothelial cells (Kawasaki J, et al. Eur J Pharmacol 1999; 373:111-20), respectively. Furthermore, the effects of troglitazone observed by Yang et al could have been attributed to another property of the troglitazone not related to its ability to activate PPARgamma or its insulin-sensitizing activity. It is surprising that, as shown below in Example 1 inhibition of IL-2 production by rosiglitazone was evident at a concentration of 0.1 micromol/L, more than twice the concentration achievable with therapeutic doses of rosiglitazone (Balfour J A, Plosker G L. Rosiglitazone. Drugs 1999; 57:921-30). This could not could not have been predicted based on the observations of Yang X Y, et al. In addition, as shown below in Example 1, the results obtained with rosiglitazone and the BP compounds (also thiazolidinediones), can be used to generate the field theory that all the inhibitory effect of troglitazone on IL-2 production is an effect representative of the entire class of tthiazolidinediones, which is also surprising.

The foregoing is offered primarily for purposes of illustration. It will be readily apparent to those of ordinary skill in the art that the operating conditions, materials, procedural steps and other parameters of the system described herein may be further modified or substituted in various ways without departing from the spirit and scope of the invention. For example, the invention has been described with human patients as the usual recipient, but veterinary use is also contemplated. Thus, the invention is not limited by the preceding description.

### EXAMPLES

### Example 1

### Method for Screening for PPARgamma Agonists or PPARgamma/PPARalpha Co-activators or PPARgamma/RXR Heterodimers, Based on Inhibition of IL-2 Production

The following test can be used to detect whether an insulin sensitizing agent is also able to inhibit IL-2 production. Isolated human T lymphocytes or a mammalian lymphocyte cell line which expresses PPARgamma is stimulated with one or a combination of PHA/PMA, TNF-alpha, interferon-gamma or some other factor that activates induction of IL-2 gene expression. Production of IL-2 is determined by measuring the concentration of IL-2 in the supernatant from cells using Endogen kits (Wolbum), as described by Yang et al. (J Biol Chem 2000;275:4541-4). Preincubation of the same cells with 5 micromolar of troglitazone or prostaglandin J2 or LG100754 for 12 hours prior to addition of an activator of IL-2 production inhibits the activation of IL-2 production otherwise observed in the absence of a thiazolidinedione.

TABLE I
Inhibition of PHA/PMA-induced IL-2 secretion in human T lymphocytes
by rosiglitazone and other thiazolidinedione PPARgamma agonists

| T lymphocytes‡ | IL-2 released | Percent Significance* | |
|---|---|---|---|
| +PHA/PMA | (pg/ml supernatant) | Inhibition | (2-tailed t-test) |
| Control (no drug) | 456 ± 28 | — | |
| 10 uM rosiglitazone | 112 ± 7 | 75 | |
| 10 uM BP1-003 | 37 ± 7 | 92 | 0.007 |
| 10 uM BP1-008 | 91 ± 6 | 80 | 0.189 |
| 10 uM BP1-017 | 37 ± 6 | 92 | 0.052 |

‡Preincubated with drug for 24 hr
*Significanly different from rosiglitazone
BP1-003: N-(2-{4-[(2,4-dioxo(1,3-thiazolidin-5-yl)methyl]phenoxy}ethyl)-5-(1,2-dithiolan-3-yl)-N-methylpentan-amide
BP1-008: dithiol derivative of BP1-003
BP1-017: dithiol derivative of BP1-003
The BP series of compounds, see US PAT. NO. 6,127,394 “1,2-Dithiolane Derivatives”

TABLE II
Dose dependent Inhibition of PHA/PMA-induced IL-2 secretion in
human T lymphocytes by rosiglitazone and BP1-0017

| T lymphocytes† | IL-2 released | Percent |
|---|---|---|
| +PHA/PMA | (c.p.m.) | Inhibition |
| Control (No drug) | 545 ± 41 | — |
| 0.1 uM Rosiglitazone | 481 ± 11 | 12 |
| 1.0 uM Rosiglitazone | 406 ± 12 | 25 |
| 10 uM Rosiglitazone | 323 ± 26 | 41 |
| 0.1 uM BP1017 | 507 ± 22 | 7 |
| 1.0 uM BP1017 | 368 ± 12 | 32 |
| 10 uM BP1017 | 160 ± 18 | 71 |

†Preincubated with drug for 12 hr

### Example 1b

### Method for Screening for PPARgamma Agonists or PPARgamma/PPARalpha Co-activators or PPARgamma/RXR Heterodimers, Based on Inhibition of NF-kappaB Activation

The following test can be used to generically identify compounds of use in this invention based upon their ability to act as an agonist of PPAR. Thiazolidinediones like troglitazone, pioglitazone and rosiglitazone are non-prostaglandin compounds that bind and activate PPARgamma. The rexinoid, LG100754 is a selective RXR/PPARgamma agonists. These compounds are tested for the ability to inhibit activity of NF-kappaB. Isolated human T lymphocytes or a mammalian cell line such as a Jurkat T cell line which expresses PPARgamma is stimulated with a concentration of one or a combination of: phytohemagglutinin/phorbol 12-myristate 13-acetate (PHA/PMA), TNF-alpha, interferon-gamma or some other factor that activates NF-kappaB. Activation of NF-kappaB is determined by electrophoretic mobility shift assay similar to that described by Rossi et al. Preincubation of the same cells with 5 micromolar of troglitazone or prostaglandin J2 or LG 100754 for 2 hours prior to addition of an activator of NF-kappaB inhibits the activation of NF-kappaB otherwise observed in the absence of a thiazolidinedione.

### Example 2a

### Measurement of in Vitro Adipocyte Differentiation Activity.

The following examples 2a and 2b provides a generic means to measure adipocyte differentation to determine if one has an insulin-sensitizing agent. A mouse preadipocyte cell line (3T3-L1) obtained from the American Type Culture Collection, and the cells are grown in a Dulbecco's modified Eagle medium (DMEM) containing 4.5 g/L glucose, 50 mg/L streptomycin sulfate, 100 000 units/L penicillin-G, 0.584 g/L L-glutamine, 4 mg/L pantothenate, 8 mg/L D-biotin, and 10 mM HEPES (pH 7.2)] supplemented with 10% fetal bovine serum (FBS). The cells are then plated at 1.5×104/cm2 in a 96-well tissue culture plate (view plate, 96 white, Packard) coated with type 1 collagen. After the cells had reached confluence, the cells were further cultured with differentiation medium DMEM supplemented with 5% FBS, 100 ng/mL insulin, 0.1 mM isobutylmethylxanthine (IBMX), and 1 mM dexamethasone, and containing various concentrations of compounds for 4 days. The compounds added from a stock solution of dimethyl sulfoxide (DMSO). The final concentration of DMSO in the differentiation medium does not exceed 0.1% (v/v). DMSO (0.1%) was added to the control cultures. The medium was replaced with maintenance medium (DMEM supplemented with 5% of FBS and 100 ng/mL of insulin), and the cells cultured for 2 more days. Activity of stimulation of adipogenesis was determined by exposure of the cells to [14-C]-acetic acid (7.4 kBq/mL), and uptake of [14-C]-acetic acid monitored after 1 h of incubation. The medium is discarded and the cells washed twice with phosphate-buffered saline. The cells are air-dried, and 200 mL of scintillation cocktail (Microscint-20, Packard) added to the wells, and counted with a Packard TopCount microplate scintillation counter. Stimulation of adipogenesis is expressed as concentrations equivalent to the [14-C] label uptake counts in the treatment with 0.2 g/mL troglitazone.

### Example 2b

### Measurement of in Vivo Insulin-Sensitivity Activity

The hypoglycemic activity of the test compounds in insulin resistant obese fatty (fa/fa) Zucker rats (Jackson Laboratory, Bar Harbor, Me.). These rats are profoundly insulin resistant with extremely high blood concentrations of insulin. Lean littennates (-/-) are used as controls. Each test compounds is administered to three Zucker rats at 10 mg/kg daily for five days after which blood samples are taken in the nonfasting state. Blood samples are collected, placed in a hematocrit centrifuge tube, and centrifuged to obtain plasma. Insulin in the collected plasma is measured by means of a radioimmuno assay kit (Linco Research, Inc, St Charles, Mo.). The insulin-sensitizing activity of the test compounds are calculated as follows:

Insulin-sensitivity activity (%)=[(PI in C−PI in T)/PI in C]×100

where “PI in C” is plasma insulin in control rats and “PI in T” is plasma insulin in rats treated with test compounds.

### Example 3

### Method for Screening for PPARgamma Agonists or PPARgamma/PPARalpha Co-activators or PPARgamma/RXR Heterodimers, Based on Inhibition of NFAT Activation

Isolated human T lymphocytes or a mammalian cell line such as a Jurkat T cell line which expresses PPARgamma is stimulated with a concentration of one or a combination of PHA/PMA, TNF-alpha, interferon-gamma or some other factor that activates NFAT. Transcriptional activation of NFAT is determined by electrophoretic mobility shift assay similar to that described by Yang et al. Preincubation of the same cells with 5 micromolar of troglitazone or prostaglandin J2 or LG100754 for 2 hours prior to addition of an activator of NFAT inhibits the activation of NFAT otherwise observed in the absence of a thiazolidinedione.

### Example 4

### An Animal Trial, Therapy for Preventing Acute and Chronic Allograft Rejection

A laboratory rat is selected for experimental renal transplantation. An allograft having moderate immunological incompatibility is selected for transplantation. The rat is given a compound that modifies the activity of PPARgamma such as a thiazolidinedione. Troglitazone or pioglitazone, 200 mg/kg daily, is given by gavage for one week pre-operatively. One kidney is excised and the rat then receives an allograft kidney transplanted from a donor rat of a different strain. Oral troglitazone or pioglitazone therapy is continued post-operatively. One to four weeks later, the rat is sacrificed and the transplanted kidney evaluated histologically for evidence of allograft rejection. The identical experiment is conducted on a control animal given placebo in place of the troglitazone or pioglitazone. Histological evidence of rejection is reduced or prevented by treatment with the troglitazone or pioglitazone

To monitor the protection by troglitazone or pioglitazone from chronic allograft rejection, the identical experiment is performed but therapy is continued for 3 to 6 months prior to sacrificing the animals.

### Example 5

### A Clinical Trial, Therapy for Preventing Acute and Chronic Allograft Rejection

A patient who is a candidate for kidney, liver or heart transplantation or other form of organ transplantation is selected for the therapy embodied in this writing. The patient may or may not be receiving other therapies for transplant rejection. A compound that modifies the activity of PPARgamma such as a thiazolidinedione (e.g. rosiglitazone) is orally administered in a dosage effective to achieve suppression of T cell activation as known to those with skill in the art. Therapy is initiated 2 weeks prior to transplantation. Within 24 to 48 hours post-operatively, thiazolidinedione (e.g., rosiglitazone) therapy is resumed and the patient is monitored for changes in symptoms and signs consistent with acute (usually occurring within days) or chronic (within 2 to 6 months) rejection, as known to a practitioner skilled in the art of managing post-transplantation allograft rejection/survival. Additionally, a complete blood count, including white cell count and differential, a platelet count, and plasma IL-2 levels, serum creatinine and blood, urea, nitrogen [BUN] levels, liver function tests (such as levels of alkaline phosphatase, lactose dehydrogenase, and transaminases), lipid profile, blood glucose, urinary protein and other tests or evaluations known to a practitioner skilled in the art of managing post-transplantation allograft rejection/survival, are checked prior to allograft transplantation, immediately post-operatively (for monitoring acute rejection) and periodically thereafter for the ensuing months, up to 6 months (for monitoring chronic rejection). Administration of the thiazolidinedione or other compound that modifies the activity of PPARgamma or PPARgamma/RXR heterodimers prevents or decreases signs or symptoms of allograft rejection. The administration of the therapy also enables the clinician to decrease the dose of other conventionally used immunosuppressive agents without increasing the risk of allograft rejection. The patient experiences fewer side effects associated with the other conventional immunosuppressive agents.

### Example 6

### Synergistic Therapy with a PPAR Agonist and a Calcineurin Inhibitor or a Macrolide Immunophilin Modulator

Immunophilin modulators such as the calcineurin inhibitors cyclosporine or tacrolimus (FK506), or the macrolide antibiotic, sirolimus are potent immunosuppresive agents. Methods of achieving synergistic effects for enhanced immunosuppression and inhibition of inflammation, proliferation, or prevention of apoptosis of cells or tissues constituting the target lesion includes the use of the aforementioned PPAR ligands in combination with a compound sufficient to bind and inhibit calcineurin, subsequently inhibiting the expression or production of inflammatory cytokines thereby ameliorating, arresting, or preventing apoptosis of the diseased cells or tissues, and preventing allograft rejection, a T lymphocyte-mediated disease involving apoptosis. A preferred dosage range for administration of cyclosporine, tacrolimus or sirolimus would typically be from 0.1 to 100 mg/m2 of body surface area, depending on the compound's ability to bind to or modify the activity of its PPARgamma and/or PPARalpha, given in single or divided doses, orally or by continuous infusion, two or three times per day. For example, for preventing allograft rejection AND ASSOCIATED COMPLICATIONS, cyclosporine is administered orally as adjunctive therapy as outlined in: DRUG FACTS AND COMPARISONS, 2000 Edition.

### Example 7

### A Clinical Trial, Synergistic Therapy for Preventing Allograft Rejection Syndrome and Complications of Concomitant Immunosuppressive Therapy

To achieve a synergistic effect, the treatment can be modified to include combination therapy with a thiazolidinedione (PPARgamma ligand) or rexinoid (e.g. LG100754, a PPARgamma/RXR heterodimer ligand) and another immunosuppressive compound traditionally used for preventing allograft rejection. Examples of such compounds that provide for synergistic effect when given in combination with the drugs encompassed by the current invention include ligands for the glucocorticoid nuclear receptor ligand (e.g. prednisone), inhibitors of purine synthesis (e.g. azathioprine and mycophenolate), calcineurin inhibitors (e.g. cyclosporine, tacrolimus), and the immunophilin modulator, sirolimus. One or a combination of these compounds are employed (at dosages described above in the section on Dosage and Schedules) in clinical trials similar to the one described above in Example 5 or in doses sufficient to prevent or treat allograft rejection. Examples of synergistic combinations are:

a. A thiazolidinedione given orally (rosiglitazone, 4 mg twice daily; or pioglitazone, 45 mg once daily; or troglitazone 300 mg twice daily) is administered in combination with prednisone.

b. A thiazolidinedione is administered in combination with prednisone and cyclosporin A or tacrolimus.

c. A thiazolidinedione is administered in combination with prednisone and cyclosporin A or tacrolimus, and azathioprine or mycophenolate.

d. A non-thiazolidinedione PPARgamma ligand (e.g. an alpha-methoxy-beta-phenyl propanoic acid derivative, an N-(2-Benzoylphenyl)-L-tyrosine derivative, a phenylacetic acid derivative or a PPARgamma-selective cyclopentenone prostaglandin in the A1 or J2 series or prostaglandin-like compound), is administered in combination with one or more FDA-approved immunosuppressive transplant rejection therapeutic compound, as described in examples a, b and c above.

e. A rexinoid PPARgamma/RXR heterodimer ligand (e.g. LG100754) is administered in combination with one or more FDA-approved immunosuppressive transplant rejection therapeutic compound, as described in examples a, b and c above.

All of the documents referred to herein are incorporated by reference as if reproduced in full below.

Methods claimed in this invention, in part, applies to synthetic PPAR ligands described in detail in the following issued, allowed, pending or provisional patent applications:

1. U.S. Pat. No. 6,127,394 1,2-Dithiolane Derivatives, pending

2. U.S. Provisional Pat. No. 60/157890 Novel Dithiolane Derivatives

3. U.S. Pat. No. 6,103,742 Pharmaceutical composition

4. U.S. Pat. No. 6,100,403 Production of benzaldehyde compounds

5. U.S. Pat. No. 6,087,385 Flavonoid derivatives

6. U.S. Pat. No. 6,087,384 Apoptosis inhibitor

7. U.S. Pat. No. 6,028,088 Flavonoid derivatives

8. U.S. Pat. No. RE36,575 Pyridine and thiazolidinedione derivatives

9. U.S. Pat. No. 6,022,897 Selective modulators of PPAR gamma and methods.

10. U.S. Pat. No. 6,011,036 Heterocyclic compounds having antidiabetic . . .

11. U.S. Pat. No. 6,011,031 Azolidinediones useful for the treatment of diabetes . . .

12. U.S. Pat. No. 6,008,237 Arylthiazolidinedione derivatives

13. U.S. Pat. No. 5,990,139 Thiazolidinedione derivatives or salts thereof and . . .

14. U.S. Pat. No. 5,985,884 Heterocyclic compounds, process for their preparation . . .

15. U.S. Pat. No. 5,977,365 Heterocyclic compound having anti-diabetic activity

16. U.S. Pat. No. 5,972,970 Oxazolidinedione derivatives, their production and use

17. U.S. Pat. No. 5,972,959 Oxime derivatives, their preparation and their therapeutic use

18. U.S. Pat. No. 5,965,589 Thiazolidinedione derivatives, their production and use

19. U.S. Pat. No. 5,962,470 Heterocyclic compounds having anti-diabetic activity . . .

20. U.S. Pat. No. 5,952,509 Production of benzaldehyde compounds

21. U.S. Pat. No. 5,965,584 Pharmaceutical composition

22. U.S. Pat. No. 5,952,356 Pharmaceutical composition

23. U.S. Pat. No. 5,939,442 Modulations of PPAR gamma, and methods for the use thereof

24. U.S. Pat. No. 5,932,601 Oxazolidinedione derivatives, their production and use

25. U.S. Pat. No. 5,925,656 Compounds having antidiabetic, hypolipidemic . . .

26. U.S. Pat. No. 5,919,782 Heterocyclic compounds having antidiabetic, hypolipidaemic . . .

27. U.S. Pat. No. 5,910,592 Substituted thiazolidinedione derivatives

28. U.S. Pat. No. 5,902,726 Activators of the nuclear orphan receptor PPARgamma

29. U.S. Pat. No. 5,889,032 Heterocyclic compounds having antidiabetic . . .

30. U.S. Pat. No. 5,889,025 Antidiabetic compounds having hypolipidaemic . . .

31. U.S. Pat. No. 5,886,014 Benzimidazole derivatives, their preparation . . .

32. U.S. Pat. No. 5,885,997 Heterocyclic compounds, process for their preparation . . .

33. U.S. Pat. No. 5,869,495 Heterocyclic compounds as pharmaceutical

34. U.S. Pat. No. 5,859,051 Antidiabetic agents

35. U.S. Pat. No. 5,847,008 Method of treating diabetes and related disease states

36. U.S. Pat. No. 5,843,970 Thiazolidine derivatives for the treatment of hypertension

37. U.S. Pat. No. 5,834,501 Heterocyclic compounds having anti-diabetic activity . . .

38. U.S. Pat. No. 5,827,865 Heterocyclic compounds as pharmaceutical

39. U.S. Pat. No. 5,824,694 Thiazolidine derivatives for the treatment of psoriasis

40. U.S. Pat. No. 5,811,439 Thiazolidinedione derivatives, method for preparing . . .

41. U.S. Pat. No. 5,801,173 Heterocyclic compounds having antidiabetic . . .

42. U.S. Pat. No. 5,741,803 Substituted thiazolidinedionle derivatives

43. U.S. Pat. No. 5,693,651 Quinoline derivatives

44. U.S. Pat. No. 5,506,245 Thiazolidinedione compounds

45. U.S. Pat. No. 6,174,904 Pharmaceutical composition

46. U.S. Pat. No. 6,172,090 Pharmaceutical composition

47. U.S. Pat. No. 6,172,089 Pharmaceutical composition

48. U.S. Pat. No. 6,169,100 Pharmaceutical composition

49. U.S. Pat. No. 6,169,099 Pharmaceutical composition

50. U.S. Pat. No. 6,166,043 Pharmaceutical composition

51. U.S. Pat. No. 6,166,042 Pharmaceutical composition

52. U.S. Pat. No. 6,156,773 Pharmaceutical composition

53. U.S. Pat. No. 6,153,632 Method and composition for the treatment of diabetes

54. U.S. Pat. No. 6,150,384 Pharmaceutical composition

55. U.S. Pat. No. 6,150,383 Pharmaceutical composition

56. U.S. Pat. No. 6,133,295 Pharmaceutical composition

57. U.S. Pat. No. 6,133,293 Pharmaceutical composition

58. U.S. Pat. No. 6,130,214 Benzothiazin and benzoxazin derivatives; their preparation and uses

59. U.S. Pat. No. 6,121,295 Pharmaceutical composition

60. U.S. Pat. No. 6,121,294 Pharmaceutical composition

61. U.S. Pat. No. 6,121,288 Visceral fat lowering agent

62. U.S. Pat. No. 6,117,893 Heterocyclic compounds having anti-diabetic activity and their use

63. U.S. Pat. No. 6,103,907 Phenylalkylcarboxylic acid compounds and compositions for treating

64. U.S. Pat. No. 6,160,000 Antidiabetic agents based on aryl and heteroarylacetic acids

65. U.S. Pat. No. 6,090,839 Antidiabetic agents

66. U.S. Pat. No. 6,090,836 Benzisoxazole-derived antidiabetic compounds

67. U.S. Pat. No. 6,080,765 Pharmaceutical composition

68. U.S. Pat. No. 6,054,453 Tricyclic compounds and their use in medicine . . .

69. U.S. Pat. No. 6,028,109 Use of agonists of the peroxisome proliferator activated receptor alpha . . .

70. U.S. Pat. No. 6,028,052 Treating NIDDM with RXR agonists

71. U.S. Pat. No. 6,020,382 Method of treating diabetes and related disease states

72. U.S. Pat. No. 5,972,881 Treating NIDDM with RXR agonists

73. U.S. Pat. No. 5,962,470 Heterocyclic compounds having anti-diabetic activity and their use

74. U.S. Pat. No. 5,780,490 Oxime derivatives, their preparation and their therapeutic use

75. U.S. Pat. No. 5,739,345 Intermediate compounds in the preparation of heterocyclic compounds

76. U.S. Pat. No. 5,703,096 Oxime derivatives, their preparation and their therapeutic use

77. U.S. Pat. No. 5,624,935 Heterocyclic compounds having anti-diabetic activity and their use

All publications, patents and patent applications mentioned in this specification are herein incorporated by reference into the specification in their entirety for all purposes.

Although the invention has been described with reference to preferred embodiments and examples thereof, the scope of the present invention is not limited only to those described embodiments. As will be apparent to persons skilled in the art, modifications and adaptations to the above-described invention can be made without departing from the spirit and scope of the invention, which is defined and circumscribed by the appended claims.

The foregoing is offered primarily for purposes of illustration. It will be readily apparent to those of ordinary skill in the art that the operating conditions, materials, procedural steps and other parameters of the invention described herein may be further modified or substituted in various ways without departing from the spirit and scope of the invention. For example, the invention has been described with human patients as the usual recipient, but veterinary use is also contemplated. Thus, the preceding description of the invention should not be viewed as limiting but as merely exemplary.

What is claimed is:

1. A method of treating the rejection of an organ or tissue or cell and promoting allograft survival, by attenuating or preventing: (i) acute rejection of the allograft, (ii) chronic rejection of the allograft, (iii) the post-transplantation syndrome complex associated with allograft rejection, and (iv) the post-transplantation syndrome complex associated with pharmacological treatments used to prevent allograft rejection, where the method comprises the step of administering to an human or animal in need of treatment an effective amount of a insulin-sensitizing agent that blocks IL-2 production, or pharmaceutically acceptable salts, solvates, tautomers or stereoisomers thereof.

2. A method of claim 1 wherein the organ tissue or cell is selected from the group consisting of: kidney, liver, heart, skin or skin appendage, lung, alimentary tract, blood vessel, bone, cartilage, nerve, muscle, cornea, retina, cochlea, exocrine gland, endocrine gland, brain tissue, spinal cord, pancreatic islet cells, or stem cells.

3. A method of claim 1 wherein the post-transplantation syndrome complex resulting from allograft rejection and/or immunosuppressive pharmacotherapy used to prevent allograft rejection, which consists of one or more clinical conditions selected from the group consisting of: post-transplantation de novo malignancy (e.g. lymphoma, epidermal cancer), osteoporosis, osteopenia, hyperlipidemia, insulin resistance, type 2 diabetes, hypertension, nephropathy, vasculopathy, atherosclerosis, cardiomyopathy, congestive heart failure, endarteritis, myocarditis, glomerulonephritis.

4. A method of claims 1, 2, and 3 wherein the insulin sensitizing agent is a PPARgamma agonist.

5. A method of claims 1, 2, and 3 wherein the insulin sensitizing agent is a PPARalpha agonist.

6. A method of claims 4 and 5 wherein the PPARgamma agonist and the PPARalpha agonist is the same compound.

7. A method of claim 6 wherein the ED50 for PPARgamma activation is within 2 orders of magnitude of the ED50 for PPARalpha activation.

8. A method of claim 1 wherein the insulin-sensitizing agent is a thiazolidinedione.

9. A method of claims 6 and 8 wherein the thiazolidinedione is a PPAR agonist.

10. A method of claims 1 and 7 wherein the thiazolidinedione selected from the group consisting of rosiglitazone, pioglitazone, MCC 555, RWJ 241947, KRP 297, NIP-221, NIP-223, CI-1 037/CS011, CLX-0921, BRL 48482, troglitazone, englitazone, and darglitazone.

11. A method of claim 8 wherein the thiazolidinedione is a 5-aryl-2,4-thiazolidinedione.

12. A method of claim 1 wherein the insulin-sensitizing agent is an isoxazolinedione.

13. A method of claim 1 wherein the insulin-sensitizing agent is an alpha-methoxy-beta-phenylpropanoic acids.

14. A method of claim 1 wherein the insulin-sensitizing agent is a N-(2-Benzoylphenyl)-L-tyrosine derivative.

15. A method of claim 1 wherein the insulin-sensitizing agent is phenylacetic acid derivative.

16. A method of claim 1 wherein the insulin-sensitizing agent is selected from the group consisting of GI262570, GW7845, L410,198, L-764486, PNU-182716, GW 409544, GW 501516, GI 181771, CLX-0901, CLX-0940 and (JTT-501).

17. A method of claim 1 wherein the insulin-sensitizing agent is an indole-based PPARgamma agonist.

18. A method of claim 1 wherein the insulin-sensitizing agent is a RXR/PPARgamma agonist.

19. A method of claim 18 wherein the RXR/PPARgamma agonist is selected from the group consisting of LG100268, LGD100324, LG100754.

20. A method of claim 1 wherein the rejection is acute.

21. A method of claim 1 wherein the rejection is chronic.

22. A method of claim 1 wherein the rejection is graft versus host disease.

23. A method of claims 1, 2 and 3 wherein the insulin-sensitizing agent is administered as an adjuvant with one or more other immunosuppressive agents.

24. A method of claim 23 wherein the insulin-sensitizing agent is rosiglitazone administered at a total daily dose of 8 mg.

25. A method of claim 23 wherein the insulin-sensitizing agent is pioglitazone administered at a total daily dose of 45 mg.

26. A method of claim 23 wherein the insulin-sensitizing agent is KRP 297 administered at a total daily dose of 10 to 30 mg.

27. A method of claim 23 wherein the insulin-sensitizing agent is MCC 555 administered at a total daily dose of 10 to 30 mg.

28. A method of claim 23 wherein the insulin-sensitizing agent is JTT-501 administered at a total daily dose of 10 to 30 mg.

29. A method of claim 23 wherein the immunosuppressive agent is a calcineurin inhibitor/immunophilin modulator, selected from the group consisting of cyclosporine (Sandimmune, Gengraf, Neoral), tacrolimus (Prograf, FK506), ASM 981.

30. A method of claim 23 wherein the immunosuppressive agent is sirolimus (RAPA, rapamycin, Rapamune), or its derivative SDZ-RAD.

31. A method of claims 23 wherein the immunosuppressive agent is a glucocorticoid.

32. A method of claims 26 wherein the immunosuppressive agent is prednisone.

33. A method of claim 23 wherein the immunosuppressive agent is a purine synthesis inhibitor, selected from the group consisting of mycophenolate mofetil (MMF, CellCept(R), azathioprine, cyclophosphamide.

34. A method of claim 23 wherein the immunosuppressive agent is an interleukin antagonist, selected from the group consisting of basiliximab, daclizumab, deoxyspergualin.

35. A method of claim 23 wherein the immunosuppressive agent is a lymphocyte-depleting agent, selected from the group consisting of antithymocyte globulin (Thymoglobulin, Lymphoglobuline), anti-CD3 antibody (OKT3).

36. A method of claim 1 wherein the immunosuppressive agent is administered at a dose lower than those used in standard protocols, or a sub-therapeutic dose in protocols not containing an insulin-sensitizing agent.
